(19) United States
(12) Patent Application Publication
(10) Pub. No.: US 2008/0234650 A1
(43) Pub. Date: Sep. 25, 2008

(54) Anchor for nonabsorbant string of tampon

(76) Inventor: Mary A. Smith, Lawrenceburg, IN (US)

Correspondence Address: William M. Sellenke, 5 Drummond St., Cincinnati, OH, 45218, US

(21) Appl. No.: 11/726,752

(22) Filed: Mar. 22, 2007

(51) Int. Cl. A61F 13/20

(52) U.S. Cl. 604/385.18

(57) ABSTRACT

The invention is a modified withdrawal string for a tampon. The modified withdrawal string is nonabsorbant or monofilament string. The string has one or more anchor filaments at one or more locations along its length. Other attachments are a closed circumference at the end of the withdrawal string as well as a zigzag near the attachment end section of the string. The string can be textured nonabsorbant string.

### FIELD OF INVENTION

The invention is concerned with withdrawal strings attached to tampons or absorbent pledgets. More specifically the invention is concerned with nonabsorbant withdrawal strings. In particular, the invention is concerned with ways of modifying nonabsorbant string to ensure that the string is anchored within the tampon.

### BACKGROUND OF INVENTION

Tampons or absorbent pledgets are commonly used to collect and sequester menstrual discharges. There is a large body of patent art concerning these very common sanitary items. The ordinary tampon has an applicator and an absorbent body with a removal string attached. Of particular concern are removal strings that are made of hydrophobic materials. Monofilament strings may be inherently superior in preventing capillary wicking action than multistranded strings. However, either may be used. A hydrophobic removal string has a significant advantage over hydrophilic strings which may be multi-stranded cotton. This is because a nonabsorbent string does not provide a ready conduit for extravaginal bacteria to be transported via wicking into the in place absorbent tampon. It is important that exogenous bacteria such as Staphylococcus aureus are not introduced to the in place tampon. Staphylococcus aureus can survive for long periods in room temperature water. If the tampon string were exposed via contact to bacterial or viral laden toilet water, organisms could be wicked via the string. Such organisms transported by wicking can reach the inner labial or vaginal walls. This warmer, blood and sloughed tissue rich environment could result in exponential growth of organisms. Certain strains of Staphylococcus aureus can cause Toxic Shock Syndrome. A hydrophobic string can reduce the risk of acquiring this syndrome. It is also possible that a small number of bacteria could replicate rapidly in a localized internal microenvironment, producing toxin which can enter the bloodstream via microabrasions in the inner labia or the vaginal walls.

The importance of hydrophobic polymers for use as a tampon withdrawal string is noted by U.S. Pat. No. 5,458,589. However, it is common experience that polymers are smooth and therefore are difficult to anchor to a second object such as the body of the tampon. The present invention will teach means of modifying nonabsorbant string to more securely anchor the withdrawal string to the absorbent tampon.

### SUMMARY OF INVENTION

The invention is a modified withdrawal string that is attached to a tampon. The modified withdrawal string is a nonabsorbant string with an tampon attachment end and an external removal end. The tampon attachment end has a friction increasing means. The friction increasing string end is attached within the body of a tampon. The modified nonabsorbent withdrawal string that is attached to a tampon may be made of monofilament string. The attachment means of the modified nonabsorbent withdrawal string include one or more anchor filaments, at one or more locations along its length. Likewise, another string attachment means may be one or more closed circumference(s) at the end of the withdrawal string. The string can be textured nonabsorbant string. A modified withdrawal string friction increasing means is a zigzag, which zigzag may be textured at the folds of the zigzag.

### BRIEF DESCRIPTION OF FIGURES

FIG. 1 shows a current art tampon.

FIG. 2 illustrations show various modifications of tampon string attachment ends to provide various friction increasing means.

### DETAILED DESCRIPTION OF FIGURES

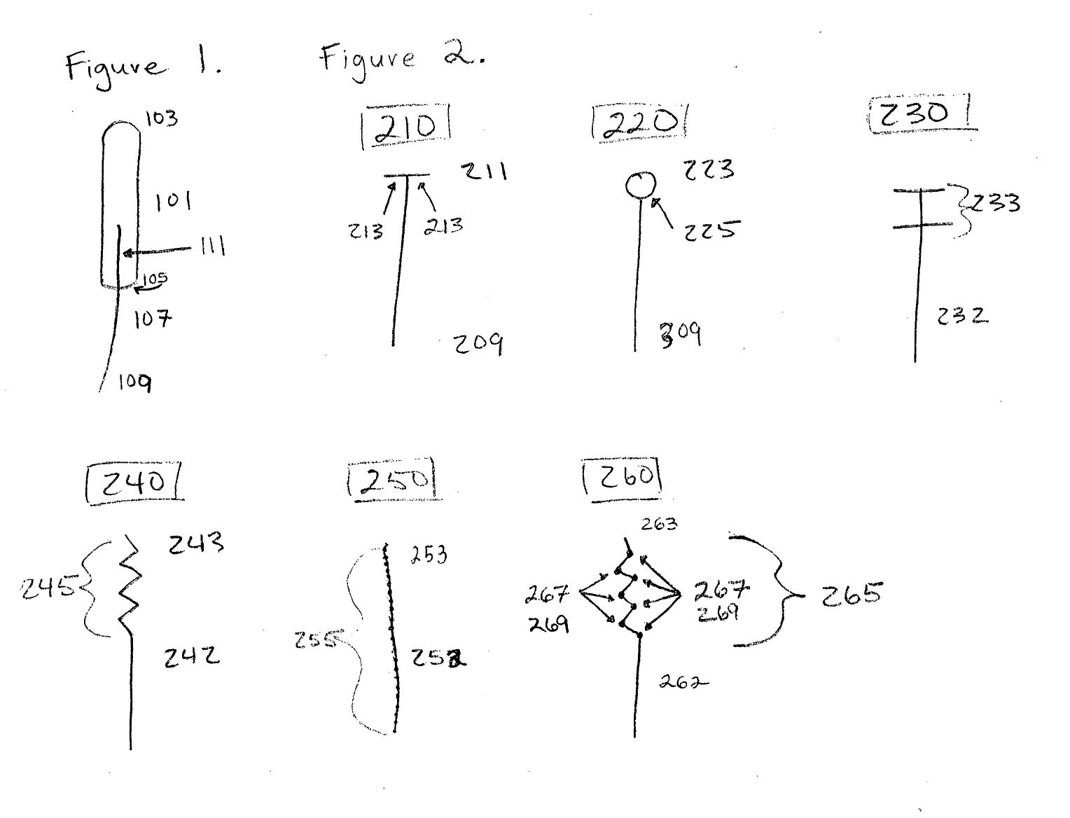

FIG. 1 shows a generic current art tampon. There is a certain variety in commercially available tampons. However, most tampons have absorbent body 101 with a rounded insertion end 103. Opposite rounded insertion end 103 is string end 105. Tampon string 107 has an attachment end 111 and an external removal end 109.

FIG. 2 illustrations 210, 220, 230, 240, 250, and 260 show various modifications of tampon string attachment ends to provide various friction increasing means. These various embodiments illustrate modifications of the body and tampon end of the withdrawal string. These modifications increase the friction or staying power of the withdrawal string within the body of the tampon. Illustration 210 shows a nonabsorbent (monofilament) tampon string with attachment end 210 and external removal end 209. Tampon string 210 has a modified attachment end 211. Attachment end 211 splits into one or more anchor filaments 213. Nonabsorbant string 220 shows that attachment end 223 ends in a close circumference or circumferences such as circle 225. The external removal end is 309

Embodiment 230 withdrawal string 232 shows multiple anchor filaments 233. Embodiment 240 shows attachment string 242 with end 243 in the form of zigzag 245. Zigzag 245 increases the resistance to slippage at attachment points of nonabsorbant string 240 within tampon absorbing body 101. Embodiment 250 shows that attachment end 253 which is placed within absorbent body 101 has textured surface 255. Textured surface 255 retains string 252 within absorbent body 101. Embodiment 260 shows attachment end 263 as a zigzag portion 265 with textured surface 267 at the folds 269 of the zigzag portion 265. Zigzag portion 265 is placed within absorbent body 101 to retain string 262 within absorbent body 101.

I claim:

1) A modified withdrawal string that is attached to a tampon comprising:
a nonabsorbant string with an tampon attachment end and an external removal end;
said tampon attachment end has a friction increasing means;
said nonabsorbant string is attached to the body of a tampon.

2) A modified withdrawal string that is attached to a tampon as in claim 1) wherein said nonabsorbent string is a monofilament string.

3) A modified withdrawal string that is attached to a tampon as in claim 1) wherein said friction increasing means further comprises one or more anchor filaments at said attachment end or at one or more locations along its length.

4) A modified withdrawal string that is attached to a tampon as in claim 1) wherein said friction increasing means is one or more closed circumference(s).

5) A modified withdrawal string that is attached to a tampon as in claim 1) wherein said friction increasing means is a zigzag.

6) A modified withdrawal string that is attached to a tampon as in claim 1) wherein said friction increasing means is textured nonabsorbant string.

7) A modified withdrawal string that is attached to a tampon as in claim 5) wherein said zigzag is textured at the folds of the zigzag.
